(19) United States
(12) Patent Application Publication
(10) Pub. No.: US 2016/0371444 A1
(43) Pub. Date: Dec. 22, 2016

(54) SYSTEM AND METHOD FOR DETERMINING AND INDICATING VALUE OF HEALTHCARE

(71) Applicant: Healthcare Value Analytics, LLC, Lufkin, TX (US)

(72) Inventor: George Fidone, Lufkin, TX (US)

(21) Appl. No.: 15/177,058

(22) Filed: Jun. 8, 2016

Related U.S. Application Data
(60) Provisional application No. 62/182,295, filed on Jun. 19, 2015.
(60) Provisional application No. 62/306,286, filed on Mar. 10, 2016.

(51) Int. Cl. G06F 19/00

(52) U.S. Cl. CPC G06F 19/328; G06F 19/327; G06F 19/322

(57) ABSTRACT

A method includes determining an average cost to treat or an expected reimbursement for a diagnosis associated with a patient. The method also includes receiving an indication of a plurality of health care services provided or scheduled for the patient. The method further includes determining a cost for each of the plurality of health care services. The method still further includes aggregating the costs for the plurality of health care services to determine a total aggregated cost. In addition, the method includes configuring, for display to a user, an indicator to indicate the total aggregated cost relative to the average cost to treat or the expected reimbursement.

### CROSS-REFERENCE TO RELATED APPLICATIONS AND CLAIM OF PRIORITY

The present application claims priority to U.S. Provisional Patent Application Ser. No. 62/182,295 filed Jun. 19, 2015, entitled “SYSTEM AND METHOD FOR DETERMINING AND INDICATING VALUE OF HEALTHCARE,” and to U.S. Provisional Patent Application Ser. No. 62/306,286 filed Mar. 10, 2016, entitled “SYSTEM AND METHOD FOR DETERMINING AND INDICATING VALUE OF HEALTH CARE.” The contents of the above-identified patent documents are incorporated herein by reference.

### TECHNICAL FIELD

This disclosure is generally directed to a system and method for determining and indicating value of health care.

### BACKGROUND

As the cost of providing health care continues to increase at an unsustainable rate, health care providers (especially physicians) are increasingly being held responsible for controlling cost in an effort to provide high quality care while controlling costs. Drivers of this shift in responsibility include the federal government (Medicare, Medicaid, VA, CHiPs), state governments, third party payers (both for-profit and not-for-profit), and Accountable Care Organizations (ACOS) as well as individual hospitals and medical groups. Increasingly, health care providers are being asked to share in the financial risk in providing medical care.

### SUMMARY

This disclosure provides a system and method for determining and real time monitoring of the value of health care being delivered in multiple settings, e.g., from the point of diagnosis through the last service provided, whether inpatient or outpatient.

In a first embodiment, a method includes determining an average cost to treat or an expected reimbursement for a diagnosis associated with a patient (e.g., a diagnosis related group (DRG), ICD, or other diagnosis method). The method also includes receiving an indication of a plurality of health care services provided, ordered, or scheduled for the patient. The method further includes determining a cost for each of the plurality of health care services. The method still further includes aggregating the costs for the plurality of health care services to determine an estimated total aggregated cost. In addition, the method includes configuring, for display to a user, an indicator to indicate the estimated total aggregated cost relative to the average cost to treat or the expected reimbursement.

In a second embodiment, an apparatus includes a memory configured to store instructions and at least one processor configured to execute the instructions. The at least one processor is configured, when executing the instructions, to determine an average cost to treat or an expected reimbursement for a diagnosis associated with a patient; receive an indication of a plurality of health care services provided, ordered, or scheduled for the patient; determine a cost for each of the plurality of health care services; aggregate the costs for the plurality of health care services to determine an estimated total aggregated cost; and configure, for display to a user, an indicator to indicate the estimated total aggregated cost relative to the average cost to treat or the expected reimbursement.

Other technical features may be readily apparent to one skilled in the art from the following figures, descriptions, and claims.

### BRIEF DESCRIPTION OF THE DRAWINGS

For a more complete understanding of this disclosure and its features, reference is now made to the following description, taken in conjunction with the accompanying drawings, in which:

FIG. 1 illustrates an example system for determining and indicating value of health care, according to this disclosure;

FIG. 2 illustrates an example device for use in the system of FIG. 1, according to this disclosure;

FIG. 3 illustrates an example display for determining and indicating value of health care that may be used in the system of FIG. 1, according to this disclosure;

FIG. 4 illustrates another example display for determining and indicating value of health care that may be used in the system of FIG. 1, according to this disclosure;

FIG. 5 illustrates another example system for determining and indicating value of health care, according to this disclosure; and

FIG. 6 illustrates an example method for determining and indicating value of health care according to this disclosure.

### DETAILED DESCRIPTION

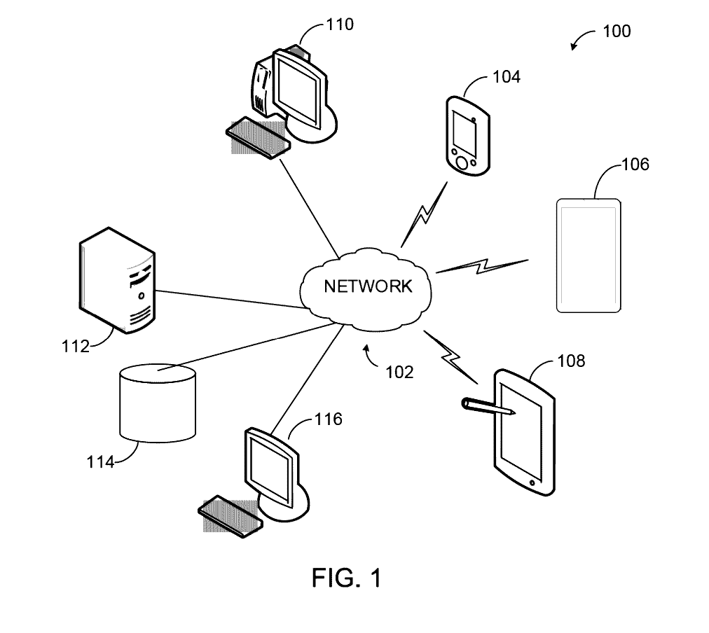

FIGS. 1 through 6, discussed below, and the various embodiments used to describe the principles of the present disclosure in this patent document are by way of illustration only and should not be construed in any way to limit the scope of this disclosure. Those skilled in the art will understand that the principles of the present disclosure may be implemented in any type of suitably arranged device or system.

Health care providers often lack information regarding the costs of services provided within a hospital or medical center. While health care providers (e.g., physicians) often are cognizant of their office charges and perhaps even their daily visit charges in a hospital setting, they typically do not know the hospital's costs associated with providing service to their patients. In addition, the providers usually do not know the average historical cost to treat a particular condition or the expected overall reimbursement that a health care facility can receive for patients being treated at the facility.

On the other hand, hospitals and other health care facilities use nationally accepted formulas for each hospitalization of a patient to arrive at an expected payment/reimbursement. Additionally, hospitals generally maintain a comprehensive charge list of the costs of materials and time for each component of care delivered. Physicians and other health care providers typically lack access to these two hospital-based components of accounting, yet these components are major drivers of the cost and value of health care delivered.

Currently, there is no mechanism for combining the available accounting information from discrete information sources for all types of care, processing this information, and then presenting the information to a provider in real-time, on-demand, in a way that helps the health care provider determine an appropriate plan of care. Current methods of reporting are incomplete, inaccurate, delayed (i.e., not real-time), and/or cumbersome (e.g., information not presented in a useful, easily readable manner).

It is therefore desirable for a provider to be able to see where the cost of care of a patient falls on a cost of care continuum, from the time of diagnosis to completion of all care delivered (e.g., a final physical therapy session six months after initial medical contact). The determination of “value” has become the new mandate from businesses, payers, and the patients themselves, each of whom look to reduce health care costs. The market is ready for a system that can determine and monitor the value of health care being delivered in multiple settings from the point of diagnosis through the last service provided, whether inpatient or outpatient.

To address these issues, embodiments of this disclosure provide systems and methods for determining and indicating value of health care. The disclosed systems and methods provide and process health care cost and value information on a regular, ongoing basis, and automatically update the information when changes are needed or desired. A provider can see where the value of the care of the patient falls within a value continuum at every input.

The disclosed systems help health care providers monitor, manage, and maximize value in the delivery of care. In some embodiments, the disclosed systems are capable of determining the average cost to treat a patient diagnosis. The systems receive information relating to a plurality of health care services associated with a patient. The systems have the ability to associate a cost with each of the health care services and the ability to aggregate the costs for the health care services. In addition, the disclosed systems are capable of presenting the service cost and value data to the health care provider in a way that it can be used to determine an appropriate plan of care for the patient.

The following embodiments are described with respect to electronically stored health care information. However, it will be clear to those of skill in the art that the disclosed embodiments are also applicable in association with other types of industries where multiple costs are aggregated from multiple components and/or multiple providers (e.g., automotive, housing, aerospace, manufacturing, industrial, etc.).

FIG. 1 illustrates an example system 100 for determining and indicating value of health care, according to an embodiment of this disclosure. The embodiment illustrated in FIG. 1 is for illustration only. Those skilled in the art will recognize that, for simplicity and clarity, some features and components are not explicitly shown, including those illustrated in connection with later figures. Such features, including those illustrated in later figures, will be understood to be equally applicable to the system 100. It will be understood that all features illustrated in the figures may be employed in any of the embodiments described. Omission of a feature or component from a particular figure is for purposes of simplicity and clarity, and not meant to imply that the feature or component cannot be employed in the embodiments described in connection with that figure.

As shown in FIG. 1, the system 100 includes a network 102. The network 102 generally represents a communication network or combination of communication networks facilitating communication between different devices or systems. Each network 102 provides any suitable communication links, such as wired, wireless, fiber optic links, or the like. In particular embodiments, the network 102 includes a combination of networks, such as the Internet, one or more cellular communication networks, and one or more local or wide area networks (which could support wired or wireless communications).

Multiple end user devices 104-110 communicate via the network 102. The user devices 104-110 generally denote devices used by health care providers or their assistants to access, provide, update, or remove information associated with patient electronic medical records (EMRs), health care costs, and health care value measurements. The user devices 104-110 include fixed or mobile devices that communicate over wired, wireless, or other connections with at least one of the networks 102.

In this example, the user devices 104-110 include a personal digital assistant 104, a smartphone 106, a tablet computer 108, and a desktop or laptop computer 110. Any other or additional user devices can be used in the system 100, and the system 100 can support interaction with any number of user devices.

One or more servers 112 also communicate over the network 102. Each server 112 represents a computing device that processes information associated with patient EMRs, health care costs, and value measurements, as described in greater detail below. Information associated with the operations of the server 112 is stored in one or more related databases 114. For example, each server 112 retrieves and provides information about one or more patient health care records, one or more medical or health care procedures associated with the patient, and cost or reimbursement information associated with the medical or health care procedures. Different information or additional information can also be provided by each server 112. Each server 112 includes any suitable structure for providing information and interacting with user devices. The database 114 includes any suitable structure for storing information and for facilitating retrieval of information (e.g., a relational database accessible through Structured Query Language (SQL) commands).

One or more operator stations 116 are capable of interacting with the server 112. For example, an operator station 116 allows health care personnel to access, provide, update, or remove information associated with patient EMRs, health care costs, and health care value measurements. Each operator station 116 includes any suitable structure supporting interaction with a server, such as a desktop computer, laptop computer, dumb terminal, or mobile device.

As described herein, each user device 104-110 executes an application or accesses an application executed by the server 112. The application allows a user to interact with, receive information from, and provide information to, the server 112. For example, the server 112 can receive requests from the user devices 104-110 and in response to receiving requests from the user devices 104-110 provides information from the database 114. Other operations supported by the application are described herein.

Although FIG. 1 illustrates one example of a system 100 for determining and indicating value of health care, various changes may be made to FIG. 1. For example, various components in FIG. 1 could be combined, further subdivided, rearranged, or omitted and additional components could be added according to particular needs.

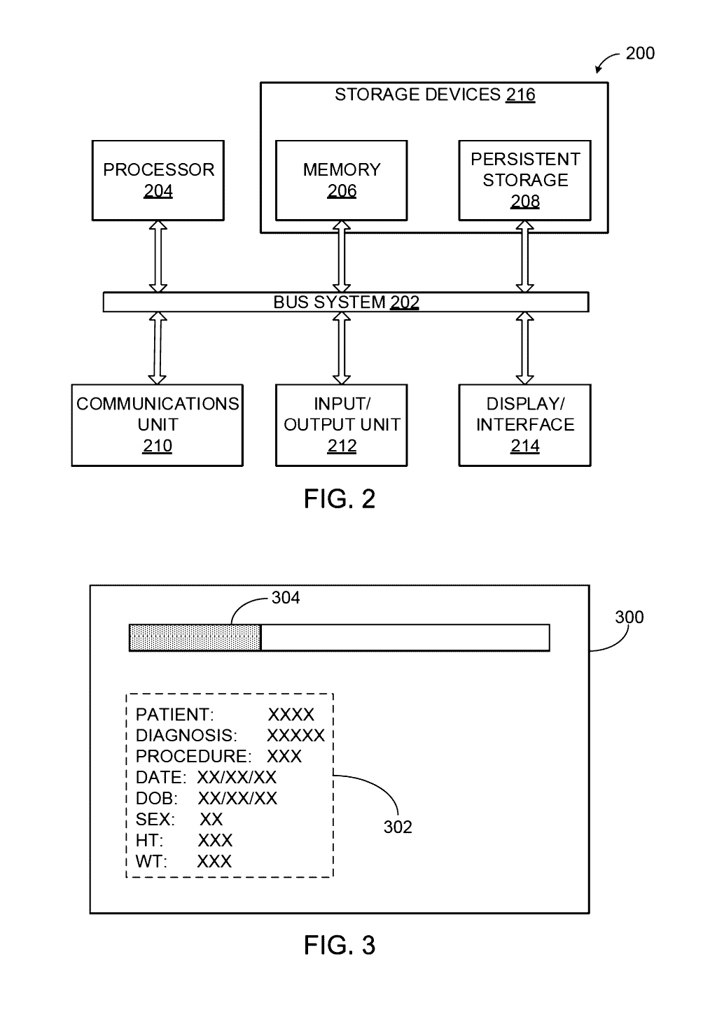

FIG. 2 illustrates an example device 200 for use in the system 100 according to this disclosure. The device 200 can represent any of the components 104-112 and 116 in FIG. 1. In this example, the device 200 includes a bus system 202. The bus system 202 supports communication between a processing device 204, a memory 206, a persistent storage 208, a communications unit 210, an input/output (I/O) unit 212, and a display or display interface 214. Any suitable bus system(s) 202 can also be used here.

The processing device 204 processes software/firmware instructions, such as instructions loaded into the memory 206. The processing device 204 can include a single processor, multiple processors, one or more multi-processor cores, or other type(s) of processor(s) depending on the particular implementation. As an example, the processing device 204 is implemented using a number of heterogeneous processor systems in which a main processor is present with secondary processors on a single chip. As another example, the processing device 204 is a symmetric multi-processor system containing multiple processors of a same or similar type. Any suitable processing device(s) can be used.

The memory 206 and the persistent storage 208 are examples of storage devices 216. A storage device is any piece of hardware capable of storing information, such as data, program code, or other suitable information on a temporary or permanent basis. The memory 206 can be a random access memory or other volatile or non-volatile storage device(s). The persistent storage 208 contains one or more components or devices, such as a hard drive, flash memory, optical disc, or other persistent storage device(s). A storage device can be fixed or removable, such as when a removable hard drive or USB thumb drive is used.

The communications unit 210 provides for communications with other systems or devices. For example, the communications unit 210 includes a network interface card or a wireless transceiver. The communications unit 210 provides communications through physical or wireless communications links.

The I/O unit 212 allows for input and output of data using other components connected to or integrated within the device 200. For example, the I/O unit 212 provides a connection for user input through a keyboard, a mouse, a microphone, or another input device. The I/O unit 212 also sends output to a display, printer, speaker, or other output device. The I/O unit 212 alternatively includes a keyboard, a mouse, a speaker, a microphone, or another input or output device(s). If the device 200 includes a display 214, the display or display interface 214 provides a mechanism to visually present information to a user. In some user devices, the display is represented as a touchscreen.

Program code for an operating system, applications, or other programs is located in the storage devices 216, which are in communication with the processing device 204 through the bus system 202. Instructions forming the programs are loaded into the memory 206 for processing by the processing device 204.

Although FIG. 2 illustrates one example of a device 200 that is used in the system 100, various changes can be made to FIG. 2. For example, FIG. 2 is simply meant to illustrate possible components in one specific implementation. Each of the components 104-112, 116 in FIG. 1 could be implemented in other ways, such as other ways that incorporate one or more processing devices, one or more memory devices storing data and instructions that are used, generated, or collected by the processing device(s), and one or more interfaces for communicating over the network 102.

As described above, the system 100 provides health care cost and value information on a regular, ongoing basis, and automatically updates the information when changes occur. Every time a provider provides care to a patient, or orders care for a patient, the provider can see how the costs of his decisions relate to the overall cost objectives for a patient with a particular diagnosis. The real-time information along with robust reporting features in the system 100 advantageously provide tools to train health care providers to be more cost-conscious in making decisions about care and to provider better value in health care.

The information can be used individually (e.g., for real-time individual decision making by a care provider) and also for comparisons between providers and provider groups (e.g., using historical reporting features). For example, for an expensive procedure (e.g., a hip replacement), one group might have providers with lower rates, but who use more expensive devices. This group could be compared against another group that charges higher rates, but has had success with less expensive devices. The information can be used to compare individual components of care (e.g., surgeon costs, device costs, and the like) and overall cost (e.g., the total cost for the hip replacement).

Additional details of the system 100 may be more readily understood by way of an example. For this example, consider a child that is admitted to a hospital for pneumonia. Before admission to the hospital, a health care provider (e.g., a physician) will have performed an examination and a work up of the patient (e.g., in the doctor's office, at an emergency care facility, or at another suitable location). Based on the exam, the physician has determined that hospitalization is needed for the patient. The physician writes admission orders to admit the patient to the hospital with a diagnosis of pneumonia. In addition to the pneumonia diagnosis, there may be co-morbidities. For example, the patient may have a supplemental oxygen requirement. Due to vomiting, the patient may be dehydrated and have low potassium. Thus, the primary diagnosis for the patient is acute pneumonia; secondary diagnoses are hypoxemia, dehydration, and hypokalemia.

Once a patient is admitted to a hospital or other health care facility, a clinical documentation improvement (CDI) specialist reviews the admitting physician's history and physical, enters the diagnoses in ICD-9 or ICD-10 codes, and executes a DRG grouper application to group the ICD-9 or ICD-10 codes and produce an initial DRG code. The CDI specialist generally begins this process upon admission. The result is known as a working DRG.

Estimated reimbursement information can be obtained from the working DRG. For example, the working DRG (the diagnoses determined from the medical record) can be multiplied by the relative weight of the DRG (a multiplier determined by the Centers for Medicare & Medicaid Services (CMS) that scores the severity of the DRG) and also multiplied by a “blended rate” (a CMS-determined multiplier that accounts for local cost influences including wage index of employees, percentage of indigent care provided, target populations, local salary and cost report information) to arrive at the expected reimbursement for that visit. The hospital also has a so-called “charge description master,” which is a list of all of the hospital's charges and costs. If the patient has further unknown conditions or complications at the time of admission, the hospital may not get any payment for treatment of those conditions.

In addition to (or in lieu of) expected reimbursement information, the diagnosis (e.g., from ICD-9 or ICD-10 codes), the working DRG, or other suitable diagnosis information can also be used to obtain expected cost information. For example, an “average cost to treat” can be obtained based on a working DRG. A baseline “average cost to treat” can be determined in advance for each DRG. These baseline “average cost to treat” values can be determined empirically by examining average total costs of treatment over time at a particular facility or group of facilities, for a particular physician group, and the like. For example, an average cost to treat for an appendectomy might be determined by examining a total cost of treatment for all appendectomies at a hospital over a two year period. In some embodiments, the average cost to treat can be determined directly from the ICD9 or ICD10 codes or other diagnosis information. In some embodiments, the average cost to treat can be determined using a neural network that includes multiple inputs, such as age, gender, ICD code(s), DRG(s), time of day, and the like.

In some embodiments, the average cost to treat can be determined based on a designed plan of care. For a particular physician group, and for a particular diagnosis, the designed plan of care may represent the authorized plan of care. The designed plan of care can include particular orders, procedures, medications, etc. These are the components that can be performed or given within the plan of care. The designed plan of care can then be used as the cost to treat baseline against which the aggregated costs are compared, as described below. The designed plan of care can be predetermined by a group of experts using evidence based medicine or clinical best practices. For example, the designed plan of care could be determined by a governmental or regulatory agency, by a corporate in-house physician group, or by any other suitable organization or group. Such designed plans of care have already been developed, reviewed, and approved for strokes, pneumonia, heart attacks, chest pain, and other common medical issues, as known in the art. The designed plan of care may include an order set that also includes target times to perform each order.

In some cases, the average cost to treat for a diagnosis may be close to the expected reimbursement for the diagnosis. In other cases, the average cost to treat may vary substantially from the expected reimbursement. Where both average cost to treat and expected reimbursement are available, both information points may be valuable to the health care provider.

All of this information is input into the system 100 (e.g., via one or more of the devices 104-110, 116) and is associated with the patient's electronic medical record(s) (which may be stored in the database 114), as described in greater detail below.

Once the patient is admitted, various costs are aggregated based on the care ordered for or provided to the patient. For example, intravenous (IV) fluids may be ordered for the patient. There is a cost to the hospital for the fluids. There is an additional cost to the hospital for nursing care associated with setting up and administering the IV fluids. As another example, an antibiotic may be ordered for the patient. There is a cost to the hospital for the antibiotic. In additional, the hospital room in which the patient stays is associated with a cost. Each of these costs is included in a running schedule or running total of costs associated with the patient's hospital stay. In conventional systems, while these costs may be predetermined, the costs may not be readily available to a care provider. In contrast, in the system 100, the orders for the IV fluids and antibiotic can be entered, and the effect of those costs for those items against the overall reimbursement or average cost to treat can be seen and evaluated before they are even administered.

The running total of costs could be indicated in the form of a progress bar across a display. For example, FIG. 3 illustrates a display 300 that may be used in the system 100. In some embodiments, the display 300 may represent a browser window in a web browser. The display 300 could display information 302 associated with the patient's electronic medical record (EMR). A value continuum progress bar 304 can be shown on the display 300 along with the information 302 in the patient's EMR after the health care provider logs into the system 100. In some embodiments, the progress bar 304 is a horizontally oriented flood bar and extends across most or all of the display 300 from the left side to the right side. In some embodiments, as the progress bar 304 “fills” from the left to the right, it changes color regions, going from green to yellow to red.

The progress bar 304 may represent a value continuum. The left (green) end of the progress bar 304 could be associated with zero cost. The right (red) end of the progress bar 304 could be associated with the expected reimbursement or average cost to treat for the medical procedure. The green region indicates that the aggregated costs of care are within the reimbursement or average cost window, and may be referred as “high value” or “premium value.” The yellow region may indicate that costs are starting to approach or exceed expected reimbursement or average cost to treat (referred to as “moderate value”). The red region may indicate that costs are exceeding expected reimbursement or average cost to treat, and the care may no longer be good value (referred to as “low value”). Thus, in general, a typical (average) patient treatment would conclude with the aggregated costs approximately in the center of the yellow region.

Each time a cost is added for the patient, the progress bar 304 is updated to reflect the additional aggregated cost. For example, when a physician orders an antibiotic for a patient, the costs associated with that care are determined, and the effect of those costs relative to the overall reimbursement or average cost to treat can be indicated in real-time by a change in the fill level of the progress bar 304 on the display 300. For example, the right edge of the progress bar 304 may extend further to the right (thereby resulting in a longer bar), and the color of the progress bar 304 may shift from green to yellow or from yellow to red. This change in the progress bar appearance can apply for all aggregated costs, including pharmacy, nursing, physical therapy, x-ray/imaging, bloodwork, and the like.

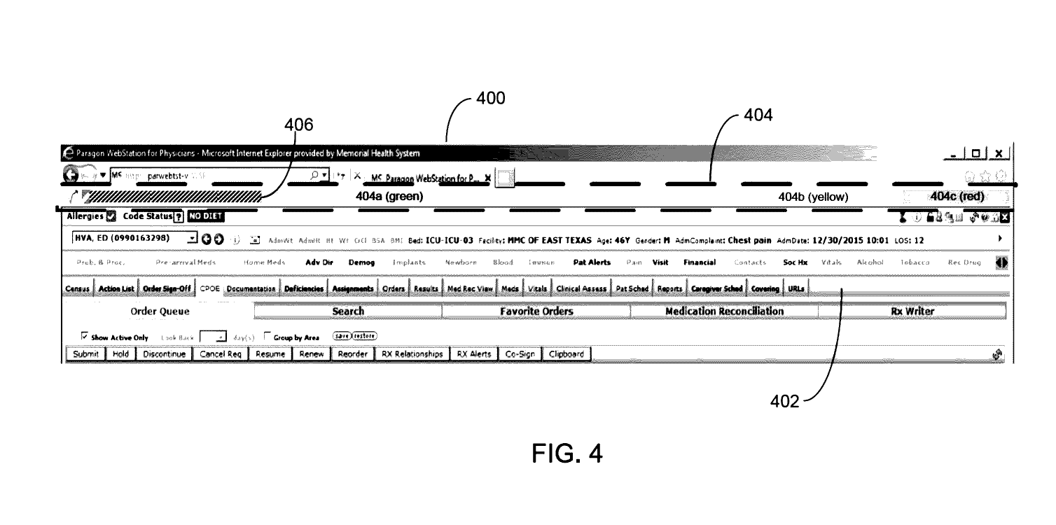

FIG. 4 illustrates another example display 400 with another example value continuum progress bar 404 that may be used in the system 100. As shown in FIG. 4, the progress bar 404 is displayed at the top of an EMR system display 402 after the health care provider logs into the EMR. In some embodiments, the progress bar 404 is integrated into (and is a part of) the EMR system display 402. In other embodiments, the progress bar 404 is independently generated and is in a separate (adjacent) window of the display 400 from the EMR system display 402. In some embodiments, the progress bar 404 only appears after a diagnosis is entered into the EMR system.

As shown in FIG. 4, the progress bar 404 includes three regions: a green region 404a, a yellow region 404b, and a red region 404c. The progress bar 404 also includes an indicator 406 that represents a current cost level on the progress bar 404. These regions 404a-404c may be similar to the green, yellow, and red regions of the progress bar 304 in FIG. 3. The background of the progress bar 404 may be scaled automatically based on the diagnosis. That is, the relative scale of each region 404a-404c and the cost thresholds associated with each region 404a-404c can be determined based on the diagnosis. For example, for one DRG (e.g., pneumonia), the transition from the green region 404a to the yellow region 404b might be associated with $6200 of aggregated costs, and the transition from the yellow region 404b to the red region 404c might be associated with $8100 of aggregated costs; for another DRG (e.g., heart surgery), the transition from the green region 404a to the yellow region 404b might be associated with $84,000 of aggregated costs, and the transition from the yellow region 404b to the red region 404c might be associated with $95,000 of aggregated costs.

In some embodiments, different aggregated or projected costs can affect the movement of the indicator 406 on the progress bar 404 at different times. For example, orders (e.g., cardiology, laboratory, medications, therapy consults, radiology, etc.) entered through a computerized physician order entry (CPOE) system can register as costs immediately, thereby causing movement of the indicator 406 to the right on the progress bar 404. As another example, scheduled procedures can register as costs on the progress bar 404 when the procedure is scheduled. As still another example, room costs can update the position of the indicator 406 on the progress bar 404 at a designated time of day, e.g., at midnight when the system 100 updates room charges.

While the physician charges and costs are accrued and billed separately, facility charges and costs are typically a direct result of physician orders. These facility charges can include:
Room (base on room type, such as private or semi-private rooms, ICU, CCU, and other)—number of nights in each room type.
Pharmacy—general, supplies, IV solutions, and other.
Pharmacy administration.
Medications requiring specific identification and requiring detailed coding.
Medical and Surgical Supplies—general devices, such as oxygen, IV solutions.
Medical and Surgical Supplies—sterile devices.
Laboratory costs—chemistry, bacteriology and microbiology, and hematology.
Operating room procedures—separate from the physician charges.
Radiology—X-ray, CAT scans, other diagnostic equipment.
Transport.
Consults.

The comparison with the overall reimbursement or average cost to treat can account for different financial factors, including profit margins, “fudge factors”, differences between insurance providers, seasonal variations of costs, and the like. For example, if an average cost to treat a particular diagnosis is $5000, but it is known that certain costs increase by approximately 10% during a particular time of year, the average cost to treat could be adjusted to $5500 before it is used as the target in the progress bar 404. Such adjustments could be made in the cost database, e.g., by a facility accountant or system administrator.

In some embodiments, the progress bar 404 can reflect an anticipated cost at discharge. The anticipated cost at discharge can be determined based on a number of factors, such as how long the patient has been admitted, the DRG, ICD9 or ICD10 codes, the costs aggregated up to a current point in time, and the like. Once determined, the anticipated cost at discharge can be displayed on or with the progress bar 404. The anticipate cost at discharge can serve as a “look ahead” feature that allows a provider to compare a current patient's costs against cost trends from similar patients so as to show how the current patient costs are tracking.

In some embodiments, the average length of stay for a procedure is also presented on the display 400, e.g., in proximity to the progress bar 404. The displayed average length of stay for a DRG can be used by the health care provider, along with the progress bar 404, to help the health care provider plan the patient care. For example, the average length of stay gives the health care provider an estimated endpoint of care, so that the provider can start to generate a discharge plan, consider or schedule resources (rooms, nurses, etc.), and the like. Also, the estimated length of stay can influence the health care provider's decisions on care. For example, if an average length of stay for an appendectomy is one night, and a particular health care provider regularly keeps patients for an average of two or three nights, the health care provider may start to change his plan of care if the provider sees on the display 400 that the average stay is one night and his standard care plan is outside the norm.

The system 100 can be used for cost comparisons. For example, if the patient experiences respiratory problems while in the hospital, the health care provider may determine that an x-ray or computerized axial tomography (CAT) scan is needed. The health care provider could provisionally enter an order for an x-ray on the display 400 and then view the movement of the progress bar 404 to see how the x-ray affects the overall value continuum. The health care provider could then provisionally enter an order for a CAT scan and then view the movement of the progress bar 404 to see how the CAT scan affects the overall value continuum. Because the cost of a CAT scan is so much higher than the cost of an x-ray, it is likely that the progress bar 404 would move closer to red due to the CAT scan than it would due to an x-ray. By comparing the movement of the progress bar 404 for each test before the test is actually ordered, the health care provider can better understand the financial impact of each test in making a determination of appropriate care.

The system 100 can also be used in early decision making by the health care provider. Consider that a health care provider creates an order that may have some cost variability. For example, an order for physical therapy can have widely varying costs, depending on the number of sessions, the progress of the therapy, the condition of the patient, and the like. In some embodiments, the system 100 can be configured to display an estimated reimbursement or an estimated average cost for the order based on the diagnosis before the order is finalized. The estimated reimbursement or average cost can be displayed in the system 100 in real time while the order is being filled to give the health care provider an indication of what an order will cost. The provider can then use those estimates in his or her decision making. As the order is filled and actual costs are incurred, they can be compared to the estimated average cost or reimbursement. In some embodiments, the actual costs can also be used to update the estimate for later procedures. Scheduled procedures can be handled in a similar manner. The average cost for a scheduled procedure can be estimated at the time the procedure is scheduled based on a historical average cost to perform the procedure on patients with the diagnosis. In situations where a DRG is not available, ICD9 or ICD10 codes could be used, or an estimated cost for a procedure could be determined based on costs for the procedure across an entire patient population or the average cost for the physician performing the procedure based on having performed the same procedure in the past.

In some embodiments, the process and metrics will be driven by the physician, since the physician is the care provider who admits patients. The physician can have an overall value metric assigned to him. The overall value metric may be based on an aggregation of overall value continuums for a plurality (e.g., some, most, or all) of the medical stays, procedures, or diagnoses for which this caregiver is the attending caregiver or caregiver in charge.

A specialist caregiver that provides services during a procedure (e.g., an anesthesiologist during a surgery) may bill separately for his professional services; thus, the fee for the specialist may not be included in the overall value continuum for a procedure. However, the resources of the facility that are used by the specialist caregiver can affect the overall value continuum. For example, one anesthesiologist may require multiple attempts to intubate, or may keep patients on a ventilator longer than other anesthesiologists, or may use more expensive anesthesia or in greater quantities than other anesthesiologists. Those costs will be incurred by the facility and will affect the overall value continuum for that procedure.

In some embodiments, consultations with other caregivers (e.g., other physicians, such as specialists) can be included in the determination of value, as shown in the overall value continuum progress bar 304. Each caregiver may have an overall value metric assigned to him. When a caregiver in charge prepares to consult another caregiver, the caregiver in charge can review the overall value metric associated with the consultant caregiver to deter mine if the consultant caregiver represents a good value. By examining overall value metrics for multiple consultant caregivers, the caregiver in charge can determine who represents the best value.

The system 100 can include one or more reporting applications or modules for reporting on value continuums. Reporting can be available to determine the overall value of each cost center (e.g., pharmacy, nurse, physician, caregiver in charge, consultant caregiver, lab, and the like) based on different parameters. The data in the reports may be grouped for a particular period of time; for a particular type of procedure, hospitalization, or diagnosis; for a particular clinic or hospital in a multi-facility hospital chain; or for any other parameter or combination of parameters. For example, reporting features can allow a user to review the overall value for all internal medicine physicians that treated pneumonia between March and September at Hospital A. The data could be broken out by physician or the data could be grouped for the physician group. Grouped data could be selected and a “drill-down” option could be applied to see more specific data. For example, grouped data may show that a physician has an average overall value for a six-month time period for pneumonia patients. However, a drill down on the grouped data may indicate that the physician had high value for all patients in the six-month period except for one patient with special circumstances, which brought the physician's average down. Trend reporting allows a user to review the value of a physician or other care provider at different points in time over a period of time.

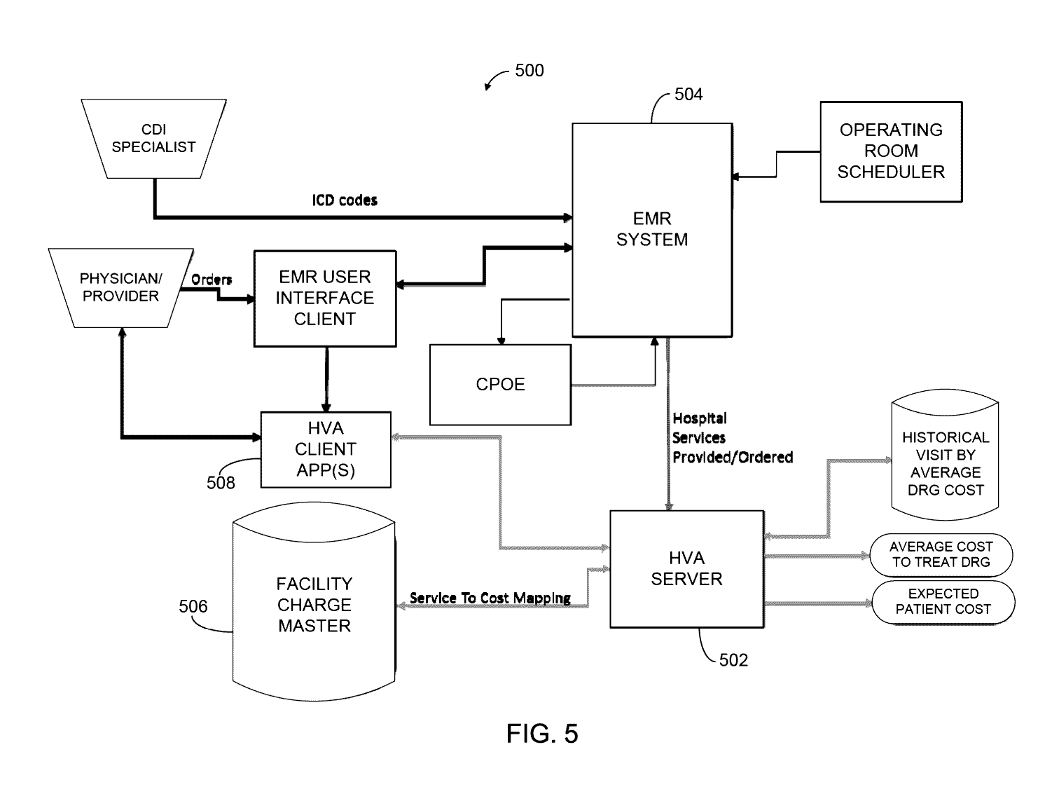

FIG. 5 illustrates another example system 500 for determining and indicating value of health care, according to an embodiment of this disclosure. One or more components of the system 500 may represent, or be represented by, one or more components of the system 100 of FIG. 1. The embodiment illustrated in FIG. 5 is for illustration only. Other embodiments could be used without departing from the scope of this disclosure.

The system 500 includes a Health Value Analytics (HVA) server 502, an EMR system 504, a facility charge master data source 506, and one or more HVA client applications 508.

The HVA server 502 is a back-end server (similar to the server 112 of FIG. 1) that connects (via a network connection) to the EMR system 504 for the hospital system or group where the system 500 is installed. The system 500 can preferably work with any EMR system 504. In some embodiments, sensitive patient or provider information is not stored in the HVA server 502, such as names, addresses, phone numbers, Social Security numbers, and personal credit card numbers or other financial data. In some embodiments, the HVA server 502 receives order information, patient information, and the like from the EMR system 504 by executing SQL commands to retrieve the information from the EMR system 504 database. In other embodiments the HVA server 502 receives order information, patient information, and the like from the EMR system 504 by an HL7 interface to the EMR system 504.

The facility charge master data source 506 is a database, data table, or other data source that includes charge information for a hospital or other facility. The HVA server 502 stores or is able to access the charge information from the facility charge master data source 506, and uses the information from the data source 506 to convert orders received from the EMR system 504 to one or more costs. In some embodiments, the facility charge master data source 506 can be received by the HVA server 502 as a file (e.g., via email). The file can then be loaded into the HVA server 502. Alternately, the HVA server 502 can connect directly to the hospital or facility accounting system to retrieve these charges. Ultimately, it is hospital or facility costs that are used in the system 500; these can be determined simply by multiplying a cost-to-charge ratio factor by the charges or have a table or method of calculating costs from the orders.

The HVA server 502 connects to a hospital or facility contracting system (e.g., PCON by MCKESSON) to obtain accurate cost data based upon admitting diagnosis codes or DRG codes when they become available. Alternatively, the HVA server 502 can calculate the cost data itself, either from a statistical model fit to historical data or from a DRG cost calculator. If the HVA server 502 performs the calculations, then factors, rates, and formulae can be stored in the HVA server 502 for easy access. If a statistical model is used, then the HVA server 502 can act upon patient symptoms or diagnosis codes for input to the model.

The HVA server 502 is configured to store one or more data stores for use by the system 500 to support model development and displays. Such data that the system 500 supports includes:
DRG average reimbursement amounts based on diagnoses, including blended rate multipliers, and other factors. This means that ICD codes are interpreted to produce the expected average reimbursement amounts.
Facility costs associated with any order that is possible for a patient. Historical data can be maintained within the system for model development, and up-to-date data can be continually updated to provide the most recent costs pertaining to the physician orders. These include: supplies (drugs, IV fluids, oxygen, and the like), producing X-rays, CAT scans, and lab work, cost of nursing care, and room charges with time increments for all types of rooms.
Any data from a facility accounting system that can help with actual reimbursements, order costs and schedule costs.

Each HVA client application 508 can be accessed on a client device or end-user device with a display (such as one of the end user devices 104-110 of FIG. 1) and provides functionality for users of the system 500 to see and check on the value continuum of care while a patient is in the hospital or facility over an admitting period of time. Each HVA client application 508 can exchange information with the HVA server 502 over a secure network link via one or more custom APIs.

Each HVA client application 508 can be installed on a client device as a stand-alone application or as a plug-in. Additionally or alternatively, each HVA client application 508 can reside on another central device (e.g., the HVA server 502) and be accessed by a client device via a portal such as a web browser.

In an embodiment, the HVA client application 508 includes a display that is the same as or similar to the display 300 of FIG. 3 or the display 400 of FIG. 4. The display can include one or more of the following features:
A progress bar (e.g., the progress bar 304, 404) that compares actual and scheduled costs aggregated by the hospital or facility against the average cost expected for care delivered or expected reimbursement based upon the admitting diagnosis or DRG codes. The progress bar can include three primary colors—Green means that the value of care is good and well within the limits expected for the average costs based on the working DRG (“premium value,” e.g., less than 85% of average cost), yellow indicates that costs are starting to approach or exceed average costs (“moderate value”), and red indicates that the value of care is poor with recognized cost overruns (“low value,” e.g., at least 115% of average cost).
Specific details that define how the progress bar is to be displayed including flood percentage and color information can be stored at the HVA server 502 and can be configurable by a user or a system administrator.
The user can click on or hover over the progress bar, which brings up additional information about the patient or about costs associated with the patient. The type of additional information that can be displayed includes but is not limited to:
   Average length of stay
   Aggregated cost of laboratory orders as compared to the average cost of laboratory orders
   Aggregated cost of radiology orders as compared to the average cost of radiology orders
   Aggregated cost for procedures as compared to the average cost of procedures
   Aggregated cost for medications ordered as compared to the average cost of medications ordered
   Aggregated cost of room charges as compared to the average cost of room charges
   Aggregated cost of therapy orders as compared to the average cost of therapy orders
   A detailed list of all the discrete orders and the associated costs for each order.
The type of additional information and the manner in which the additional information appears are configurable by a user or a system administrator.
The HVA client application 508 can interface with EMR system 504 or an order system that the physician uses to connect to the EMR system 504 (e.g., CPOE) as the care plan is updated, so that real-time updates can be sent to and from the EMR system 504 and the user can see these updates at the HVA client application 508 as the updates come through.
Information to populate the progress bar in the HVA client application 508 is provided by the HVA server 502. Calculations and display information can be controlled at the HVA server 502 or can be performed by the client device.
Security features support secure access to the HVA client application 508. For example, access to the HVA client application 508 may only be granted after a user provides a suitable user ID and password. In some embodiments, a system administrator can access a security maintenance application on the HVA server 502 to provide authorization for a user to be able to use the HVA client application 508 and see content. Since the progress bar can be displayed as part of the patient EMR view, the HVA client application 508 can infer user privileges based on corresponding user privileges in the EMR system 504. In some embodiments, the HVA client application 508 will only display the progress bar if the user is a physician of record for the patient being viewed.

The system 500 operates in real-time or nearly real-time, such that changes to data in various components of the system 500 should be reflected in other components concurrently or within a short period after the change. For example, whenever data from the EMR system 504 for a patient record that is currently on display at the HVA client application 508 is updated with new information relating to either the DRG or the orders/costs associated with the patient, the system 500 becomes aware of the data changes within a short period (e.g., 15 seconds) of the change and presents updated HVA value content on the display of the HVA client application 508.

In some embodiments, when the HVA client application 508 displays comparison data for a patient, the HVA client application 508 can display data comparisons in one of several different modes. The activity surrounding the patient during the displayed admission cycle can cause the progress bar to move from green to yellow to red. The user can select the type of comparison he/she wishes to see, including:
Actual and scheduled charges compared to the average cost to treat for the facility.
The actual and scheduled charges compared against a peer group of physicians treating similar patients. The patients could be categorized as similar based on the patients having the same diagnoses codes (e.g., DRGs, ICDs, neural network, and the like). Additionally or alternatively, the patients could be categorized as similar based on demographic data (e.g., age, gender, location, and the like) or any other suitable grouping or shared characteristic. The peer group for a physician can be recognized by the system 500 based on other users of the system 500. The comparison costs come from the most recent patient admissions seen by the peer group.
The actual and scheduled charges compared against physicians covering an entire region for the same diagnoses codes. The region for a physician can be recognized by the system 500 based on other users of the system 500. The comparison costs come from the most recent patient admissions seen by other users of the system 500.
The actual and scheduled charges compared against physicians of the same specialty treating for the same diagnoses codes. The same specialty group for a physician can be recognized by the system 500 based on other users of the system 500. The comparison costs come from the most recent patient admissions seen by the specialty group.
The actual and scheduled charges compared against a single physician's aggregated cost statistics for a group of patients over a time period.

In some embodiments, the system 500 can provide feedback on orders that are placed for a patient. The feedback can relate to the practices of other physicians treating patients with the same or similar diagnosis. For example, if a physician orders an x-ray for a patient, the system 500 can inform the physician how frequently that x-ray order is associated with treating that diagnosis. In some embodiments, for orders that are very common with the patient's diagnosis, the system 500 might not generate an alert, but for orders that are comparatively rare (e.g., other physicians order the x-ray less than 5% of the time), the system 500 generates an alert. The alert may be displayed at the HVA client application 508, and may be similar to the following: “For patients with this diagnosis, the x-ray order is only seen in 3% of the treatment plans.” Similarly, the system 500 can provide feedback on orders based on best practice treatment protocols for a diagnosis. Some hospitals, facilities, and provider groups have developed best practice treatment protocols for each diagnosis. In such cases, the system 500 can provide an alert if an order is outside the predetermined protocol.

In some embodiments, when a patient is discharged, a communication can be sent to the HVA server 502 so that an end of stay can be recorded for the patient. Once this event occurs, further communications from the EMR system 504 may not be needed for this patient. The system 500 can maintain a persistent HVA value analysis for a discharged patient (e.g., at a database in the HVA server 502).

The progress bar is displayed in the HVA client application 508 based on the expected reimbursement or average cost to the hospital or facility to treat the patient; this cost is based on the diagnosis for the patient. It is common for the DRG to change throughout the facility stay period. In such cases, the progress bar displayed in the HVA client application 508 can be updated to reflect the change whenever a new DRG code is received for a patient. Similarly, the average cost to treat may be modified at one or more points during a patient stay based on how the patient progresses, the outcome of one or more procedures, and the like.

In some cases, a working DRG code and illness severity may not be available right away when a patient is admitted to the hospital or facility. In fact, it may be many hours or even days later before even a working DRG code is known. In some embodiments, the HVA client application 508 can indicate that no DRG is currently available for this patient. In some embodiments, the system 500 can use a generic baseline DRG or a baseline diagnosis that is discerned from initial evaluation of patient symptoms.

Because some payment reform models are trending toward a single lump sum payment to a facility that includes health care provider fees, in some embodiments, the average cost to treat can include both facility costs and health care provider (e.g., physician) fees. Additionally, from that single payment, physicians and other health care workers may need to negotiate their fees with the facility. The system 500 could facilitate a “share in savings” model where the physician fees are based on the value (cost versus reimbursement) the physician provided to the patient.

Although FIG. 5 illustrates one example of a system 500 for determining and indicating value of health care, various changes may be made to FIG. 5. For example, various components in FIG. 5 could be combined, further subdivided, rearranged, or omitted and additional components could be added according to particular needs.

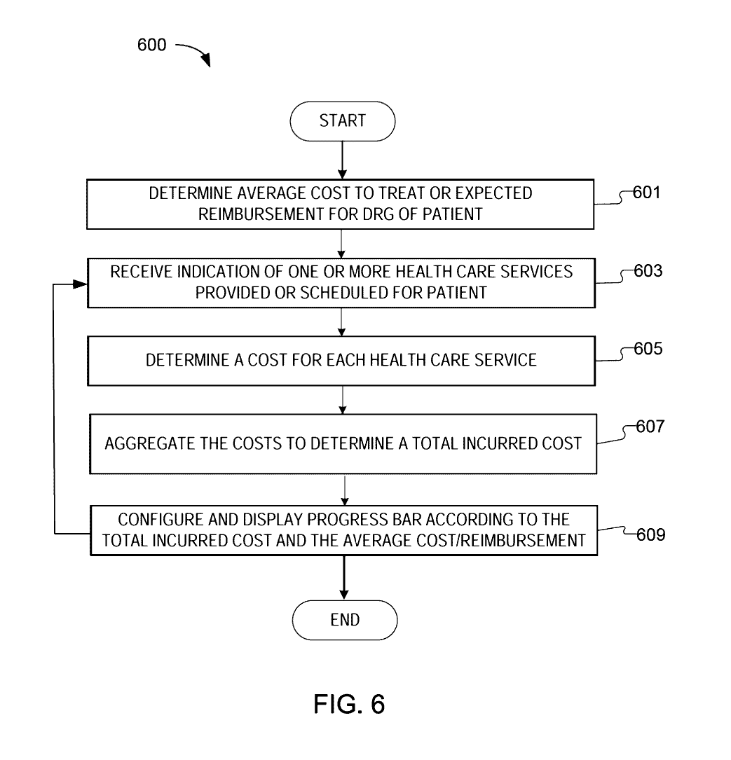

FIG. 6 illustrates an example method 600 for determining and indicating value of health care according to this disclosure. For ease of explanation, the method 600 is described as being performed using the system 500 of FIG. 5. However, the method 600 could be used with any suitable device or system.

At step 601, the system 500 determines an average cost to treat, an expected reimbursement, or both, for a DRG associated with a patient. This may include, for example, the HVA server 502 calculating the average cost to treat based on an average cost of treatment for all patients having the same diagnosis that are treated by a predetermined group of physicians (e.g., the physicians affiliated with the health care facility) over a predetermined historical time period (e.g., the two previous years) or at a predetermined group of health care facilities over a predetermined historical time period. Additionally or alternatively, this may include the HVA server 502 calculating the average cost to treat based on a designed plan of care for the DRG.

At step 603, the system 500 receives an indication of one or more health care services provided or scheduled for the patient. This may include, for example, a health care provider (e.g., a physician) entering one or more orders into the CPOE system and then the HVA server 502 receiving the order or an indication of the order from the EMR system 504.

At step 605, the system 500 determines a cost for each of the health care services. This may include, for example, the HVA server 502 obtaining the cost(s) from the facility charge master 506 or calculating the cost(s) based on information from the facility charge master 506.

At step 607, the system 500 then aggregates the costs for the health care services to determine a total aggregated cost. This may include, for example, the HVA server 502 adding each of the individual costs together.

At step 609, the system 500 configures an indicator to indicate the total aggregated cost relative to the average cost to treat or the expected reimbursement, and then displays the indicator to a user. This may include, for example, the HVA server 502 configuring a progress bar, such as the progress bar 304 or the progress bar 404. Once the progress bar is configured for display, a client application, such as the HVA client application 508 can display the progress bar.

Steps 603-609 can be repeated as the health care provider enters new orders or new costs are aggregated. All of the operations described in the method 600 can be performed in real-time in order to provide a real-time display to a user as patient orders are entered and costs are aggregated.

Although FIG. 6 illustrates one example of a method 600 for determining and indicating value of health care, various changes may be made to FIG. 6. For example, while shown as a series of steps, various steps shown in FIG. 6 could overlap, occur in parallel, occur in a different order, or occur multiple times. Moreover, some steps could be combined or removed and additional steps could be added according to particular needs. In addition, while the method 600 is described with respect to the system 500, the method 600 may be used in conjunction with other types of devices and systems.

The disclosed embodiments are also particularly useful for pre-paid medical services, including, but not limited to, health maintenance organizations (HMOs) and clinically integrated networks (CINs). In these environments, the disclosed progress bar can track costs for a defined period of time against a contracted pre-paid amount. As services are deployed and resources are consumed, a health care provider can quickly see exactly how the provider is performing in the value of care continuum for each patient with prepaid services.

As described above, embodiments of this disclosure provide the ability to link the cost side of patient care at the point of the provider with the reimbursement side of the patient care from the third party payer. Once the expected reimbursement is determined, that information will be entered into the system. The disclosed embodiments also provide the ability to link actual costs with estimated average costs to treat. As the primary driver of cost, the physician or other health care provider is able to monitor in relative terms or in precise terms the cost of his care during each phase of the care continuum. By linking the physician with these financial components, improved awareness and greater value for care provided will result in finally bending the cost of the health care curve downward.

In some embodiments, various functions described above are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “application” and “program” refer to one or more computer programs, software components, sets of instructions, procedures, functions, objects, classes, instances, related data, or a portion thereof adapted for implementation in a suitable computer code (including source code, object code, or executable code). The terms “transmit” and “receive,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A method comprising:
determining an average cost to treat or an expected reimbursement for a diagnosis associated with a patient;
receiving an indication of a plurality of health care services provided or scheduled for the patient;
determining a cost for each of the plurality of health care services;
aggregating the costs for the plurality of health care services to determine a total aggregated cost; and
configuring, for display to a user, an indicator to indicate the total aggregated cost relative to the average cost to treat or the expected reimbursement.

2. The method of claim 1, further comprising:
receiving an indication of at least one additional health care service provided or scheduled for the patient;
determining a cost for each of the at least one additional health care service;
adding the cost to the total aggregated cost to determine an updated total aggregated cost; and
updating the indicator to indicate the updated total aggregated cost relative to the average cost to treat or the expected reimbursement.

3. The method of claim 2, wherein the receiving, determining, configuring, and updating operations are performed in real-time to provide a real-time display to the user.

4. The method of claim 2, wherein the indication of the plurality of health care services or the indication of the at least one additional health care service is received from an electronic medical record (EMR) system after a user enters an order in a computerized physician order entry (CPOE) system.

5. The method of claim 2, wherein the cost for each of the plurality of health care services or the at least one additional health care service is determined based on charge information from a facility charge master.

6. The method of claim 1, wherein configuring the indicator comprises configuring a flood bar that has a plurality of predetermined thresholds based on the average cost to treat or the expected reimbursement, wherein a current level of the flood bar is associated with the aggregate costs for the plurality of health care services.

7. The method of claim 6, wherein the flood bar comprises:
a first region associated with high value;
a second region associated with moderate value; and
a third region associated with low value,
wherein the current level of the flood bar within one of the first, second, or third region indicates a relative value of the plurality of health care services.

8. The method of claim 1, wherein the average cost to treat is based on an average cost of treatment for the diagnosis by a predetermined group of physicians over a predetermined historical time period.

9. The method of claim 1, wherein the average cost to treat is based on an average cost of treatment for the diagnosis at a predetermined group of health care facilities over a predetermined historical time period.

10. The method of claim 1, wherein the average cost to treat is based on a designed plan of care for the diagnosis.

11. An apparatus comprising:
a memory configured to store instructions;
at least one processor configured, when executing the instructions, to:
determine an average cost to treat or an expected reimbursement for a diagnosis associated with a patient;
receive an indication of a plurality of health care services provided or scheduled for the patient;
determine a cost for each of the plurality of health care services;
aggregate the costs for the plurality of health care services to determine a total aggregated cost; and
configure, for display to a user, an indicator to indicate the total aggregated cost relative to the average cost to treat or the expected reimbursement.

12. The apparatus of claim 11, wherein the at least one processor is further configured to:
receive an indication of at least one additional health care service provided or scheduled for the patient;
determine a cost for each of the at least one additional health care service;
add the cost to the total aggregated cost to determine an updated total aggregated cost; and
update the indicator to indicate the updated total aggregated cost relative to the average cost to treat or the expected reimbursement.

13. The apparatus of claim 12, wherein the at least one processor is configured to perform the receive, determine, configure, and update operations in real-time to provide a real-time display to the user.

14. The apparatus of claim 12, wherein the at least one processor is configured to receive the indication of the plurality of health care services or the indication of the at least one additional health care from an electronic medical record (EMR) system after a user enters an order in a computerized physician order entry (CPOE) system.

15. The apparatus of claim 12, wherein the at least one processor is configured to determine the cost for each of the plurality of health care services or the at least one additional health care service based on charge information from a facility charge master.

16. The apparatus of claim 11, wherein the at least one processor is configured to scale the indicator by configuring a flood bar that has a plurality of predetermined thresholds based on the average cost to treat or the expected reimbursement, wherein a current level of the flood bar is associated with the aggregate costs for the plurality of health care services.

17. The apparatus of claim 16, wherein the flood bar comprises:
a first region associated with high value;
a second region associated with moderate value; and
a third region associated with low value,
wherein the current level of the flood bar within one of the first, second, or third region indicates a relative value of the plurality of health care services.

18. The apparatus of claim 11, wherein the average cost to treat is based on an average cost of treatment for the diagnosis by a predetermined group of physicians over a predetermined historical time period.

19. The apparatus of claim 11, wherein the average cost to treat is based on an average cost of treatment for the diagnosis at a predetermined group of health care facilities over a predetermined historical time period.

20. The apparatus of claim 11, wherein the average cost to treat is based on a designed plan of care for the diagnosis.
